(19) United States
(12) Patent Application Publication
(10) Pub. No.: US 2019/0054103 A1
(43) Pub. Date: Feb. 21, 2019

(54) METHOD OF TREATMENT OF TP53 WILD-TYPE TUMORS WITH 2',2'-DIFLUORO-5-AZA-2'-DEOXYCYTIDINE OR PRODRUGS THEREOF

(71) Applicant: EPIGENETICS PHARMA, LLC, Mercier Island, WA (US)

(72) Inventor: Richard DAIFUKU, Mercier Island, WA (US)

(21) Appl. No.: 16/079,268

(22) PCT Filed: Feb. 24, 2017
(86) PCT No.: PCT/CA2017/050242

§ 371 Date: Aug. 23, 2018

Related U.S. Application Data
(60) Provisional application No. 62/300,655, filed on Feb. 26, 2016.

(51) Int. Cl. A61K 31/706

(52) U.S. Cl. CPC A61K 31/706

(57) ABSTRACT

This disclosure provides methods and strategies for inhibiting the growth of TP53 wild-type cancer cells, comprising contacting the cell with 2′,2′-difluoro-5-aza-2′-deoxycytidine or a 2′,2′-difluoro-5-aza-2′-deoxycytidine prodrug. Also provided are related methods for treating a cancer characterized by having wild-type TP53 in a subject in need thereof.

### CROSS-REFERENCE TO RELATED APPLICATION

This application claims the benefit of US provisional application 62/300,655 filed on Feb. 26, 2016, the disclosure of which is hereby incorporated by reference in its entirety.

### TECHNICAL FIELD

Aberrant DNA methylation is an epigenetic mechanism that can inactivate the expression of genes that suppress tumorigenesis. The genes involved include tumor suppressor genes; genes that suppress apoptosis, metastasis and angiogenesis; genes that repair DNA; and genes that express tumor-associated antigens. The molecular mechanism of silencing gene expression appears to be due to the attachment of 5-methylcytosine binding proteins to the methylated promoter, which blocks the action of transcription factors.

Because this epigenetic change is reversible, it presents a promising target for chemotherapeutic intervention. For example, the tumor suppressor gene TP53 is of vital importance in preventing human cancer development and progression and is often referred to as the “guardian of the genome.” Mutations of the encoding gene are detected in approximately 50% of all types of human cancers (so called TP53 null or mutant). The remaining cancers that remain wild-type for TP53 (TP53 WT) often possess alterations that effect the expression, stability, and/or functionality of the associated p53 protein, such as post-translational mechanisms and epigenetic modifications such as DNA methylation. Through these modifications, p53 protein is often inactivated even in TP53 WT cancers because the p53 protein can otherwise trigger cell growth arrest, apoptosis, utophagy, or senescence, which are all detrimental to cancer cells, and the p53 protein also impedes cell migration, metabolism, or angiogenesis, which would otherwise be favorable to cancer cell progression and metastasis. Given that TP53 can accrue mutations over time, thus, enhancing the aggressiveness of a cancer via inactive p53 protein, TP53 WT cancers represent a useful target for early intervention therapies.

Several pyrimidine analogs have been tested for the treatment of a neoplasm, and more particularly cancer. 5-Azacytidine was the first hypomethylating agent approved by the FDA for the treatment of a neoplasm and the deoxy-analog, 5-azadeoxycytidine or decitabine, was approved shortly thereafter for the same indication, which are directed to preventing or reversing methylation of tumor suppressor genes such as TP53. Both drugs produce remissions or clinical improvements in more than half of the treated patients with myelodysplastic syndrome (MDS). Features of responses include the requirement for multiple cycles of therapy, slow responses, and actual clonal elimination. Optimization of therapy has included (1) reducing the dose to favor hypomethylation over cytotoxicity, (2) prolonging administration schedules, and (3) increasing dose intensity without reaching cytotoxicity. Molecularly, hypomethylation and gene reactivation have been shown and seem to be required for responses. The data in MDS represent a proof-of-principle for epigenetic therapy. Although the therapy is effective, with complete responses lasting months to years in some patients, resistance seems to develop in the majority of patients, and the mechanisms of resistance are unknown. However, it has been reported that decitabine induces more apoptosis in TP53 null cells than in TP53 WT cells. These results pose the hypothesis that partial inhibition of DNA methylation by decitabine may cause lethality for TP53 deficient cells but not WT cells.

Gemcitabine is approved as first line treatment for pancreatic cancer and as combination therapy for other solid tumors. It is an inhibitor of DNA synthesis and, most relevant to NUC013, gemcitabine diphosphate inhibits ribonucleotide reductase (RNR). Inhibition of RNR causes a reduction in the concentrations of deoxynucleotides, including deoxycytidine triphosphate (dCTP). As gemcitabine triphosphate competes with dCTP for incorporation into DNA, the reduction in the intracellular concentration of dCTP enhances the incorporation of gemcitabine triphosphate into DNA (a mechanism that has been described as self-potentiation).

Despite the advances in the art, there remains a need for early intervention strategies that address the inactivation of tumor suppressor genes by ameliorating the effects of deleterious epigenetic modifications in cancer cells. The present disclosure addresses this and related needs.

### SUMMARY

This summary is provided to introduce a selection of embodiments that are further described below in the Detailed Description. This summary is not intended to identify key features of the claimed subject matter, nor is it intended to be used as the sole section for determining the scope of the claimed subject matter. Additional objects and features of the present compounds, compositions, methods and uses will become more apparent upon reading of the following non-restrictive description of exemplary embodiments, which should not be interpreted as limiting the scope of the disclosure.

In one embodiment is provided a method for inhibiting the growth of a TP53 wild-type cancer cell, comprising contacting the TP53 wild-type cancer cell with 2′,2′-difluoro-5-aza-2′-deoxycytidine or a 2′,2′-difluoro-5-aza-2′-deoxycytidine prodrug or a composition comprising 2′,2′-difluoro-5-aza-2′-deoxycytidine or a 2′,2′-difluoro-5-aza-2′-deoxycytidine prodrug and a pharmaceutically acceptable carrier, diluent or excipient.

In one embodiment is provided the method described therein, wherein the 2′,2′-difluoro-5-aza-2′-deoxycytidine prodrug is a silylated compound of 2′,2′-difluoro-5-aza-2′-deoxycytidine or the 2′,2′-difluoro-5-aza-2′-deoxycytidine prodrug.

In one embodiment is provided the method described therein, wherein the 2′,2′-difluoro-5-aza-2′-deoxycytidine prodrug is 3′,5′-di(trimethylsylyl)-2′,2′-difluoro-aza-2′-deoxycytidine.

In one embodiment is provided the method described therein, wherein the 2′,2′-difluoro-5-aza-2′-deoxycytidine prodrug is a tocopherol phosphate prodrug of 2′,2′-difluoro-5-aza-2′-deoxycytidine.

In one embodiment is provided the method described therein, wherein the prodrug is a tocotrienol phosphate prodrug of 2′,2′-difluoro-5-aza-2′-deoxycytidine.

In one embodiment is provided the method described therein, wherein the cancer cell is from a solid tumor, preferably a solid tumor carcinoma. In one embodiment, the solid tumor carcinoma is selected from non-small cell lung NSCL, colon, renal, central nervous system CNS, melanoma, ovarian, prostate, pancreatic, and breast carcinomas.

In one embodiment is provided the method described therein, wherein the cancer cell is from a hematologic malignancy, preferably leukemia, lymphoma, or multiple myeloma. In one embodiment is provided the method described therein, wherein the cell is in in vitro.

In one embodiment is provided the method described therein, wherein the cell is in vivo in a subject and an effective amount of the 2′,2′-difluoro-5-aza-2′-deoxycytidine or a 2′,2′-difluoro-5-aza-2′-deoxycytidine prodrug is administered to the subject, preferably the subject is human.

A method of treating a cancer characterized by having wild-type TP53, comprising administering to a subject in need thereof a therapeutically effective amount of 2′,2′-difluoro-5-aza-2′-deoxycytidine or a 2′,2′-difluoro-5-aza-2′-deoxycytidine prodrug or a composition comprising 2′,2′-difluoro-5-aza-2′-deoxycytidine or a 2′,2′-difluoro-5-aza-2′-deoxycytidine prodrug and a pharmaceutically acceptable carrier, diluent or excipient.

In one embodiment is provided the method described therein, wherein the 2′,2′-difluoro-5-aza-2′-deoxycytidine prodrug is a silylated compound of 2′,2′-difluoro-5-aza-2′-deoxycytidine or the 2′,2′-difluoro-5-aza-2′-deoxycytidine prodrug.

In one embodiment is provided the method described therein, wherein the 2′,2′-difluoro-5-aza-2′-deoxycytidine prodrug is 3′,5′-di(trimethylsylyl)-2′,2′-difluoro-aza-2′-deoxycytidine.

In one embodiment is provided the method described therein, wherein the 2′,2′-difluoro-5-aza-2′-deoxycytidine prodrug is a tocopherol phosphate prodrug of 2′,2′-difluoro-5-aza-2′-deoxycytidine.

In one embodiment is provided the method described therein, wherein the prodrug is a tocotrienol phosphate prodrug of 2′,2′-difluoro-5-aza-2′-deoxycytidine.

In one embodiment is provided the method described therein, wherein the cancer is a solid tumor, preferably a solid tumor carcinoma. In one embodiment, the solid tumor carcinoma is selected from non-small cell lung NSCLC, colon, renal, central nervous system CNS, melanoma, ovarian, renal, prostate, pancreatic, and breast carcinomas.

In one embodiment is provided the method described therein, wherein the cancer is a hematologic malignancy, preferably leukemia, lymphoma, or multiple myeloma.

In one embodiment is provided the method described therein, wherein the subject is a human.

In one embodiment is provided the use of 2′,2′-difluoro-5-aza-2′-deoxycytidine or a 2′,2′-difluoro-5-aza-2′-deoxycytidine prodrug or a composition comprising 2′,2′-difluoro-5-aza-2′-deoxycytidine or a 2′,2′-difluoro-5-aza-2′-deoxycytidine prodrug and a pharmaceutically acceptable carrier, diluent or excipient for inhibiting the growth of a TP53 wild-type cancer cell.

In one embodiment is provided the use described therein, wherein the 2′,2′-difluoro-5-aza-2′-deoxycytidine prodrug is a silylated compound of 2′,2′-difluoro-5-aza-2′-deoxycytidine or the 2′,2′-difluoro-5-aza-2′-deoxycytidine prodrug.

In one embodiment is provided the use described therein, wherein the 2′,2′-difluoro-5-aza-2′-deoxycytidine prodrug is 3′,5′-di(trimethylsylyl)-2′,2′-difluoro-aza-2′-deoxycytidine.

In one embodiment is provided the use described therein, wherein the 2′,2′-difluoro-5-aza-2′-deoxycytidine prodrug is a tocopherol phosphate prodrug of 2′,2′-difluoro-5-aza-2′-deoxycytidine.

In one embodiment is provided the use described therein, wherein the prodrug is a tocotrienol phosphate prodrug of 2′,2′-difluoro-5-aza-2′-deoxycytidine.

In one embodiment is provided the use described therein, wherein the cancer cell is from a solid tumor, preferably a solid tumor carcinoma. In one embodiment, the solid tumor carcinoma is selected from non-small cell lung NSCL, colon, renal, central nervous system CNS, melanoma, ovarian, prostate, pancreatic, and breast carcinomas.

In one embodiment is provided the use described therein, wherein the cancer cell is from a hematologic malignancy, preferably leukemia, lymphoma, or multiple myeloma.

In one embodiment is provided the use described therein, wherein the cell is in vitro.

In one embodiment is provided the use described therein, wherein the cell is in vivo in a subject and an effective amount of the 2′,2′-difluoro-5-aza-2′-deoxycytidine or a 2′,2′-difluoro-5-aza-2′-deoxycytidine prodrug is administered to the subject, preferably the subject is human.

In one embodiment is provided the use of a therapeutically effective amount of 2′,2′-difluoro-5-aza-2′-deoxycytidine or a 2′,2′-difluoro-5-aza-2′-deoxycytidine prodrug or a composition comprising 2′,2′-difluoro-5-aza-2′-deoxycytidine or a 2′,2′-difluoro-5-aza-2′-deoxycytidine prodrug and a pharmaceutically acceptable carrier, diluent or excipient for treating a cancer characterized by having wild-type TP53.

In one embodiment is provided the use described therein, wherein the 2′,2′-difluoro-5-aza-2′-deoxycytidine prodrug is a silylated compound of 2′,2′-difluoro-5-aza-2′-deoxycytidine or the 2′,2′-difluoro-5-aza-2′-deoxycytidine prodrug.

In one embodiment is provided the use described therein, wherein the 2′,2′-difluoro-5-aza-2′-deoxycytidine prodrug is 3′,5′-di(trimethylsylyl)-2′,2′-difluoro-aza-2′-deoxycytidine.

In one embodiment is provided the use described therein, wherein the 2′,2′-difluoro-5-aza-2′-deoxycytidine prodrug is a tocopherol phosphate prodrug of 2′,2′-difluoro-5-aza-2′-deoxycytidine.

In one embodiment is provided the use described therein, wherein the prodrug is a tocotrienol phosphate prodrug of 2′,2′-difluoro-5-aza-2′-deoxycytidine.

In one embodiment is provided the use described therein, wherein the cancer is a solid tumor, preferably a solid tumor carcinoma.

In one embodiment is provided the use described therein, wherein the solid tumor carcinoma is selected from non-small cell lung NSCLC, colon, renal, central nervous system CNS, melanoma, ovarian, renal, prostate, pancreatic, and breast carcinomas.

In one embodiment is provided the use described therein, wherein the cancer is a hematologic malignancy, preferably leukemia, lymphoma, or multiple myeloma.

In one embodiment is provided the use described therein, wherein the subject is a human.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 graphically illustrates a comparison of growth inhibition of TP53 WT colon cancer cell line, Ls174T, resulting from decitabine or NUC013.

FIG. 2 graphically illustrates a comparison of growth inhibition of TP53 WT colon cancer cell line LoVo resulting from decitabine or NUC013.

FIG. 3 illustrates the tumor volume as a function of time since tumor implant. The first data point on each graph is on the day of study drug treatment initiation. Values are ± standard deviation. A. HL-60 comparison of NUC013 20 mg/kg vs saline control. B. LoVo comparison of NUC013 20 mg/kg and 40 mg/kg vs saline control.

### DETAILED DESCRIPTION

The present disclosure provides therapeutic strategies for addressing TP53 wild-type cancers using a fluorinated pyrimidine analog, 2′,2′-difluoro-5-aza-2′-deoxycytidine (also referred to as “NUC013”), or prodrug versions thereof. This disclosure is based on the surprising discovery that while NUC013 is significantly more active as the related pyrimidine analog decitabine in the treatment of many TP53 null/mutant cells, NUC013 can be even more so in the treatment of TP53 wild-type cells. Furthermore, studies indicate that NUC013 and its prodrug NUC041 can be less toxic than decitabine in a xenograft model of TP53 WT cancer, further evidencing the surprising benefits of NUC013 over similar compositions.

Accordingly, in one aspect, the present disclosure provides a method for inhibiting the growth of a TP53 wild-type cancer cell, comprising contacting the TP53 wild-type cancer cell with 2′,2′-difluoro-5-aza-2′-deoxycytidine or a 2′,2′-difluoro-5-aza-2′-deoxycytidine prodrug.

### TP53 Wild-Type Cancer Cell

As used herein, the term “TP53 wild-type” or “TP53 WT” refers to a cell that contains one or more genes with a sequence encoding the wild-type p53 protein. In one embodiment, TP53 WT refers to a cell that has at least one gene with a sequence encoding the wild-type p53 protein. In another embodiment, TP53 WT refers to a cell that is homozygous for genes with sequences encoding the wild-type p53 protein.

The status of a cell for its TP53 gene sequence can be readily determined by a person of skill in the art using known techniques. Sequences for the TP53 gene, or its homologs, are known. For example, in humans, a sequence of an exemplary wild-type TP53 transcript is set forth in Genbank entry NM_001276761, incorporated herein by reference in its entirety. A “TP53 null” is a cell that comprises a mutated or other sequence variant of the TP53 gene that results in lack of expression of p53, while “TP53 mutant” cell comprises the expression of a mutated or other variant. As indicated herein, approximately 50% of all types of human cancers have mutations in the TP53 gene (TP53 null/mutant). In TP53 WT cancers, although the cells have at least one gene encoding the WT p53 protein, the gene may be silenced by, for example, DNA methylation or the functions and stability of the p53 protein can be abrogated via, for example, post-translational mechanisms. p53 protein is often inactivated in cancer considering its role in cell growth arrest, apoptosis, utophagy, or senescence, and it impedes cell migration, metabolism, or angiogenesis, which are detrimental to cancer cells and their ability to progress and/or metastasize. See, e.g., Zhang Q, et al., “Targeting p53-MDM2-MDMX loop for cancer therapy.” Subcell Biochem. 85:281-319 (2014), incorporated herein by reference in its entirety.

2′,2′-difluoro-5-aza-2′-deoxycytidine and Prodrug Forms

2′,2′-difluoro-5-aza-2′-deoxycytidine, also referred to herein as “NUC013”, is a DNA methyl transferase inhibitor (DNMTI) comprised of the base of 5-azacytidines and the sugar moiety of gemcitabine. In the case of the two FDA approved 5-azacytidines, the sugar is unmodified, and is either ribose in the case of 5-azacytidine or deoxyribose in the case of 5-aza-2′-deoxycytidine or decitabine. Both compounds have been shown to be DNMTIs. To be active as a DNMTI, 5-azacytidine needs to be reduced to 5-aza-2′-deoxycytidine by ribonucleotide reductase (RNR). 5-aza-2′-deoxycytidine has to be phosphorylated by kinases to 5-aza-2′-deoxycytidine triphosphate, which can then be incorporated into DNA, wherein it can inactivate DNMT by donating a formyl group for covalent linkage to the active site of the enzyme, resulting in depletion of DNMT. See Saunthararajah Y. “Key clinical observations after 5-azacytidine and decitabine treatment of myelodysplastic syndromes suggest practical solutions for better outcomes.” Hematology Am Soc Hematol Educ Program. 2013:511-21 (2013) and Christman J K., “5-azacytidine and 5-aza-2′-deoxycytidine as inhibitors of DNA methylation: mechanistic studies and their implication for cancer therapy.” Oncogene. 21:5483-5495 (2002), each of which is incorporated herein by reference in its entirety. On the other hand, gemcitabine (2′,2′-difluoro-2′-deoxycytine) is comprised of a modified difluorinated sugar but has an unmodified cytosine base. To be biologically active, gemcitabine needs to be converted by kinases to gemcitabine diphosphate and gemcitabine triphosphate. Gemcitabine has a number of different biological activities but of most interest in the present context is the inhibition of RNR. This inhibition increases the likelihood of gemcitabine incorporation in DNA by decreasing the competition for incorporation into DNA by deoxycytidine triphosphates. See Mini E, et al., “Cellular pharmacology of gemcitabine.” Ann Oncol. 17 (Suppl 5):v7-12 (2006), incorporated by reference in its entirety. As described in more detail below, it is demonstrated that the bonding of the 5-azacytosine base from 5-azacytidines with the 2′,2′-difluorinated sugar from gemcitabine in the same compound (i.e., NUC013) is likely responsible for activity that neither drug (i.e., azacytidines or gemcitabine) could provide, whether alone or in a combined administration.

A description of an illustrative strategy to synthesize 2′,2′-difluoro-5-azadeoxycytidine (NUC013) is provided in Example 1 below. Initial characterization of 2′,2′-difluoro-5-azadeoxycytidine is described in U.S. Patent Publication No. 2014/0024612, incorporated herein by reference in its entirety.

Anomers and prodrugs of NUC013 are also contemplated for use in the present methods. Various prodrugs of NUC013 have been described. See, e.g., WO2014/143051, describing silylated prodrugs, and WO2016/057825 describing vitamin E-based prodrugs of NUC013, each of which is incorporated by reference in its entirety.

In certain embodiments, the methods of the disclosure advantageously utilize NUC013 prodrugs. In one embodiment, representative useful NUC013 prodrugs include silylated prodrugs and vitamin E prodrugs that can have improved pharmacokinetic properties compared to NUC013 itself.

In one embodiment, useful silylated prodrugs include NUC013 analogs having a silyl group (e.g., —SiR₃) attached at one or more hydroxyl positions of the ribose sugar or the amino group of the base. Representative silylated prodrugs are described below.

In one embodiment, the silylated prodrug has the formula:

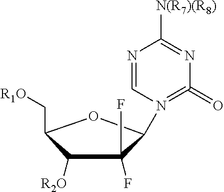

wherein R₁, R₂, R₇, and R₈ are independently selected from hydrogen or Si(R₄)(R₅)(R₆), wherein R₄, R₅, and R₆ are independently selected from hydrogen or alkyl, provided that at least one of R₁, R₂, R₇, or R₈ is Si(R₄)(R₅)(R₆). In one embodiment, R₁ is Si(R₃)(R₄)(R₅) and R₂ is hydrogen. In another embodiment, R₁ is hydrogen and R₂ is Si(R₃)(R₄)(R₅). In a further embodiment, R₁ and R₂ are Si(R₃)(R₄)(R₅). In one embodiment, R₃, R₄, and R₅ are methyl.

In another embodiment, the silylated prodrug has the formula:

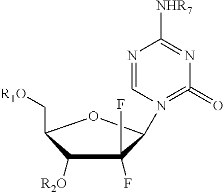

wherein R₁, R₂, and R₇ are independently selected from hydrogen or Si(R₄)(R₅)(R₆), wherein R₄, R₅, and R₆ are independently selected from hydrogen or alkyl, provided that at least one of R₁, R₂, or R₇ is Si(R₄)(R₅)(R₆). In one embodiment, R₁ is Si(R₃)(R₄)(R₅) and R₂ is hydrogen. In another embodiment, R₁ is hydrogen and R₂ is Si(R₃)(R₄)(R₅). In a further embodiment, R₁ and R₂ are Si(R₃)(R₄)(R₅). In one embodiment, R₃, R₄, and R₅ are methyl.

In a further embodiment, the silylated prodrug has the formula:

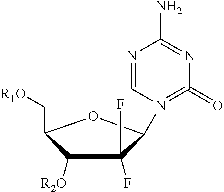

wherein R₁ and R₂ are independently selected from hydrogen or Si(R₄)(R₅)(R₆), wherein R₄, R₅, and R₆ are independently selected from hydrogen or alkyl, provided that at least one of R₁ or R₂ is Si(R₄)(R₅)(R₆). In one embodiment, R₁ is Si(R₃)(R₄)(R₅) and R₂ is hydrogen. In another embodiment, R₁ is hydrogen and R₂ is Si(R₃)(R₄)(R₅). In a further embodiment, R₁ and R₂ are Si(R₃)(R₄)(R₅). In one embodiment, R₃, R₄, and R₅ are methyl.

As used herein, the term “alkyl” refers to the alkyl group of the trialkylsilyl group and is a C1-C10 alkyl group (e.g., methyl, ethyl, n-propyl, i-propyl, n-butyl, s-butyl, i-butyl, t-butyl) that may be a straight chain, branched, or cyclic alkyl group. In certain embodiments, the alkyl group is a C1-C6 alkyl group. In certain embodiments, the alkyl group is a C1-C4 alkyl group. In certain specific embodiments, the alkyl group is a methyl group. In certain embodiments, each alkyl group of the trialkylsilyl group is the same (e.g., trimethylsilyl). In other embodiments, the trialkylsilyl group includes two or three different alkyl groups (e.g., t-butyldimethylsilyl).

Useful vitamin E prodrugs include NUC013 analogs that are conjugated to a vitamin E derivative via a phosphate ester or a phosphoramidate linkage.

Vitamin E prodrugs useful in the methods have Formula (I)

Y-L-Nu (I)

wherein Y is a tocopherol moiety or a tocotrienol moiety;

L is a phosphate ester or phosphoramidate linker; and

Nu is NUC013.

In some embodiments, Y is a vitamin E moiety. For example, Y can be tocopherol moiety. In some embodiments, Y is a tocotrienol moiety.

In one embodiment, the vitamin E prodrug has Formula (II):

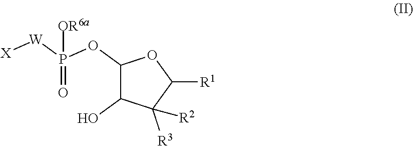

or a pharmaceutically acceptable salt thereof, wherein:

R¹ is

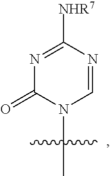

wherein R⁷ is H or C₁₋₆ alkyl;

R² and R³ are fluoro;

R^{6a} is selected from absent, H and C₁₋₆ alkyl;

W is O or NR^{6b}, wherein R^{6b} is selected from H, C₁₋₆ alkyl, C₁₋₆ alkoxy, wherein said C₁₋₆ alkyl and C₁₋₆ alkoxy are each optionally substituted with 1 or 2 substituents independently selected from aryl and heteroaryl, wherein said aryl or heteroaryl is optionally substituted with 1 or 2 substituents independently selected from cyano and nitro; and

X is

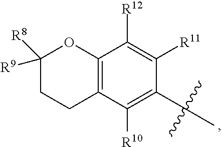

wherein

R₈ is selected from C₁₂₋₂₄ alkyl, C₁₂₋₂₄ alkenyl, C₁₂₋₂₄haloalkyl, and C₁₂₋₂₄ haloalkenyl, and R⁹, R¹⁰, R¹¹, and R¹² are each independently selected from H, C₁₋₆ alkyl, and halo.

In some embodiments, W is O. In some embodiments, W is NR^{6b}.

In some embodiments, R^{6b} is H or alkyl.

In some embodiments, R^{6a} is absent. In some embodiments, when R^{6a} is absent, W is O.

In some embodiments, R^{6a} is absent or H. In some embodiments, R^{6a} is absent or C₁₋₆ alkyl. It is understood that when R^{6a} is absent, the adjacent oxygen is negatively charged (i.e.,

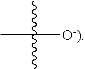

In some embodiments, R^{6a} is H or alkyl. In some embodiments, R^{6a} is H, methyl, or ethyl. In some embodiments, R^{6a} is H or methyl. In some embodiments, R^{6a} and R^{6b} are each independently H, methyl, or ethyl. In some embodiments, R^{6a} and R^{6b} are each independently H or methyl. In some embodiments, R^{6a} and R^{6b} are each H.

In some embodiments, R⁷ is H, methyl, or ethyl. In some embodiments, R⁷ is H or methyl. In some embodiments, R⁷ is H.

In some embodiments, R⁹, R¹⁰, R¹¹, and R¹² are each methyl. In some embodiments, R⁹ and R¹² are each methyl, and R¹⁰ and R¹¹ are each H. In some embodiments, R⁹, R¹¹, and R¹² are each methyl, and R¹⁰ is H. In some embodiments, R⁹, R¹⁰, and R¹² are each methyl, and R¹¹ is H.

In some embodiments, R⁸ is selected from C₁₆ alkyl, C₁₆ alkenyl, C₁₆ haloalkyl, and C₁₆ haloalkenyl. In some embodiments, R⁸ is C₁₆ alkyl or C₁₆ alkenyl.

In some embodiments, X is selected from α-tocopheryl, β-tocopheryl, γ-tocopheryl, δ-tocopheryl, α-tocotrienyl, β-tocotrienyl, γ-tocotrienyl, and δ-tocotrienyl. In certain embodiments, X is selected from

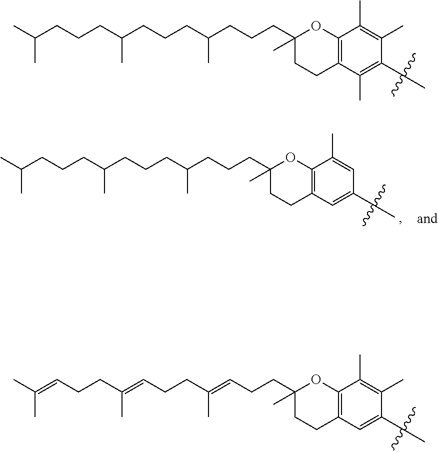 [SMILES: *c1c(C)c(C)c2c(c1C)CCC(C)(CCCC(C)CCCC(C)CCCC(C)C)O2 ; *c1cc(C)c2c(c1)CCC(C)(CCCC(C)CCCC(C)CCCC(C)C)O2 ; *c1cc2c(c(C)c1C)OC(C)(CC/C=C(\C)CC/C=C(\C)CCC=C(C)C)CC2]

In certain embodiments, the vitamin prodrug is selected from:

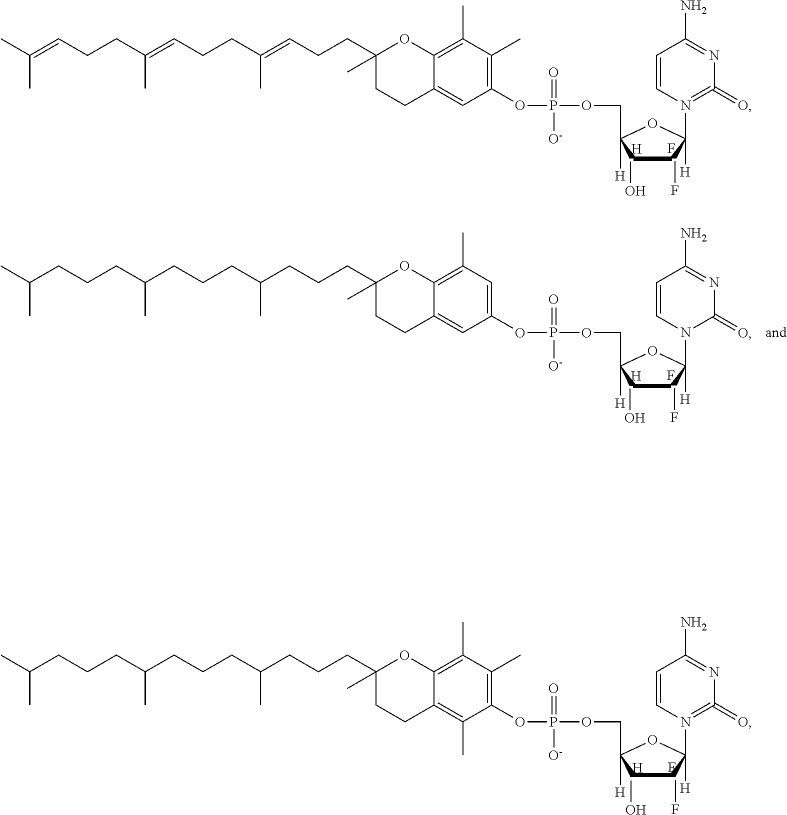

or a pharmaceutically acceptable salt thereof.

All technical and scientific terms used herein have the same meaning as commonly understood by one ordinary skilled in the art to which the present technology pertains. For convenience, the meaning of certain terms and phrases used herein are provided below. To the extent that the definitions of terms in the publications, patents, and patent applications incorporated herein by reference are contrary to the definitions set forth in this specification, the definitions in this specification control. The section headings used herein are for organizational purposes only, and are not to be construed as limiting the subject matter disclosed. The terminology used herein is for the purpose of describing particular embodiments only and is not intended to be limiting. It should be noted that, the singular forms “a”, “an”, and “the” include plural forms as well, unless the content clearly dictates otherwise. Thus, for example, reference to a composition containing “a compound” also contemplates a mixture of two or more compounds. It should also be noted that the term “or” is generally employed in its sense including “and/or” unless the content clearly dictates otherwise. Furthermore, to the extent that the terms “including”, “includes”, “having”, “has”, “with”, or variants thereof are used in either the detailed description and/or the claims, such terms are intended to be inclusive in a manner similar to the term “comprising”.

The expression “pharmaceutically acceptable prodrugs” as used herein refers to those prodrugs of the compounds formed by the process of the present description which are, within the scope of sound medical judgment, suitable for use in contact with the tissues of humans and lower animals with undue toxicity, irritation, allergic response, and the like, commensurate with a reasonable benefit/risk ratio, and effective for their intended use. “Prodrug”, as used herein means a compound which is convertible in vivo by metabolic means (e.g. by hydrolysis) to afford any compound delineated by the formulae of the instant description. Various forms of prodrugs are known in the art.

As used herein, the term “alkyl” is meant to refer to a saturated hydrocarbon group which is straight-chained (e.g., linear) or branched. Example alkyl groups include methyl (Me), ethyl (Et), propyl (e.g., n-propyl and isopropyl), butyl (e.g., n-butyl, isobutyl, t-butyl), pentyl (e.g., n-pentyl, isopentyl, neopentyl), and the like. An alkyl group can contain from 1 to about 30, from 1 to about 24, from 2 to about 24, from 1 to about 20, from 2 to about 20, from 1 to about 10, from 1 to about 8, from 1 to about 6, from 1 to about 4, or from 1 to about 3 carbon atoms.

As used herein, the term “alkylene” refers to a linking alkyl group.

As used herein, “alkenyl” refers to an alkyl group having one or more double carbon-carbon bonds. The alkenyl group can be linear or branched. Example alkenyl groups include ethenyl, propenyl, and the like. An alkenyl group can contain from 2 to about 30, from 2 to about 24, from 2 to about 20, from 2 to about 10, from 2 to about 8, from 2 to about 6, or from 2 to about 4 carbon atoms.

As used herein, “alkenylene” refers to a linking alkenyl group.

As used herein, “haloalkyl” refers to an alkyl group having one or more halogen substituents. Example haloalkyl groups include CF₃, C₂F₅, CHF₂, CCl₃, CHCl₂, C₂Cl₅, and the like.

As used herein, “haloalkylene” refers to a linking haloalkyl group.

As used herein, “haloalkenyl” refers to an alkenyl group having one or more halogen substituents.

As used herein, “haloalkenylene” refers to a linking haloalkenyl group.

In some embodiments, the prodrug is 3′,5′-di(trimethylsilyl)-2′,2′-difluoro-aza-2′-deoxycytidine. In some embodiments, the prodrug is a tocopherol phosphate prodrug of 2′,2′-difluoro-5-aza-2′-deoxycytidine. In some embodiments, the prodrug is a tocotrienol phosphate prodrug of 2′,2′-difluoro-5-aza-2′-deoxycytidine.

Thus, in this aspect, the 2′,2′-difluoro-5-aza-2′-deoxycytidine (NUC013) or a 2′,2′-difluoro-5-aza-2′-deoxycytidine prodrug is contacted to a TP53 wild-type cancer cell.

In one embodiment, there is provided a pharmaceutical composition, comprising a compound as defined in the present application, together with a pharmaceutically acceptable carrier, diluent or excipient.

A further aspect relates to the use of a compound as defined in the present application, or such a compound for use, in the treatment or prevention of a disease or condition described therein. Similarly, this aspect relates to the use of a compound of the present application in the manufacture of a medicament for the treatment or prevention of a disease or condition described therein. This aspect also further relates to a method for treating a disease or condition described therein, which comprises administering to a subject in need thereof, a therapeutically effective amount of a compound as herein defined.

Cancer Cells

In some embodiments, the cancer cell is from a solid tumor. For example, the solid tumor can be a non-small cell lung, colon, renal, central nervous system (CNS), melanoma, ovarian, pancreatic, prostate, or breast carcinoma. Given the broad-spectrum activity of NUC013, as described below, the present disclosure can also encompass embodiments where the solid tumor can be an endometrial, thyroid, esophageal, stomach, rectum, liver, bladder, testis, bone, SLC, other carcinomas or other tumors.

In some embodiments, the cancer is from a hematologic malignancy. For example, in some embodiments, the cancer is a leukemia, lymphoma, or multiple myeloma.

The method can be performed in vitro, such as in a method comprising contacting the TP53 wild-type cancer cell that is in a cell culture. The cell can be from an established multigenerational cell culture line or be a cell that has been obtained from a subject, such as by biopsy or in a biological sample. Methods, conditions, and reagents for maintaining a cancer cell in culture are known and within the abilities of persons of ordinary skill in the art. This facet of the disclosure can be used to ascertain the responsiveness or characterize the effect of NUC013 on the cell.

Additionally, the method of this aspect can be performed in vivo in a subject that has a TP53 WT cancer. An effective amount of the 2′,2′-difluoro-5-aza-2′-deoxycytidine or a 2′,2′-difluoro-5-aza-2′-deoxycytidine prodrug is administered to the subject accordingly to known and acceptable protocols for such a purpose. The 2′,2′-difluoro-5-aza-2′-deoxycytidine or a 2′,2′-difluoro-5-aza-2′-deoxycytidine prodrug can be formulated and dosed as appropriate according to known methods for any appropriate mode of delivery.

Treating Cancer

In another aspect, the disclosure provides a method of treating a cancer characterized by expressing wild-type TP53. The method comprises administering to a subject in need a composition comprising a therapeutically effective amount of 2′,2′-difluoro-5-aza-2′-deoxycytidine or a 2′,2′-difluoro-5-aza-2′-deoxycytidine prodrug.

As used herein, the term “therapeutically effective” refers to any quality, such as a dosing amount or form, which results in any improvement in the condition of the subject that results from the cancer. The therapeutic effect can include obtaining a preventative or prophylactic effect, or any alleviation of the severity of signs and symptoms of the condition, which can be detected by means of physical examination, laboratory or instrumental methods.

As used herein, the term “treating a cancer”, or similar language, refers to inhibiting the disease, disorder, and/or condition related to the presence of the cancer in the subject, such as slowing or arresting its development or progression; and/or relieving the disease, disorder or condition, e.g., causing regression of the disease, disorder and/or condition. The progress can be ascertained by comparing to development in absence of the treatment.

In some embodiments, the cancer is a solid tumor. Examples of solid tumors encompassed by the disclosure are described herein. In some embodiments, the cancer is a hematologic malignancy. Examples of hematologic malignancy encompassed by the disclosure are described herein.

The compound of the disclosure such as 2′,2′-difluoro-5-aza-2′-deoxycytidine or a 2′,2′-difluoro-5-aza-2′-deoxycytidine prodrug can be formulated for any appropriate route of administration, as is well understood in the art, such as compositions, emulsion formulations, microemulsion formulations, or micelle formulations.

In some embodiments, the compound of the disclosure such as the 2′,2′-difluoro-5-aza-2′-deoxycytidine or a 2′,2′-difluoro-5-aza-2′-deoxycytidine prodrug is formulated in a composition that also comprises an acceptable carrier, diluent, excipient etc. Preferably, the carrier, diluent or excipient is pharmaceutically acceptable.

Therapeutically effective amounts of the compounds will generally range up to the maximally tolerated dosage, but the concentrations are not critical and may vary widely. The precise amounts employed by the attending physician will vary, of course, depending on the compound, route of administration, physical condition of the patient and other factors. The daily dosage may be administered as a single dosage or may be divided into multiple doses for administration.

The amount of the compound actually administered will be a therapeutically effective amount, which term is used herein to denote the amount needed to produce a substantial beneficial effect. Effective doses may be extrapolated from dose-response curves derived from in vitro or animal model test systems. The animal model is also typically used to determine a desirable dosage range and route of administration. Such information can then be used to determine useful doses and routes for administration in humans or other mammals. The determination of an effective dose is well within the capability of those skilled in the art. Thus, the amount actually administered will be dependent upon the individual to which treatment is to be applied, and will preferably be an optimized amount such that the desired effect is achieved without significant side-effects.

Therapeutic efficacy and possible toxicity of the compounds of the disclosure can be determined by standard pharmaceutical procedures, in cell cultures or experimental animals (e.g., ED50, the dose therapeutically effective in 50% of the population; and LD50, the dose lethal to 50% of the population). The dose ratio between therapeutic and toxic effects is the therapeutic index, and it can be expressed as the ratio LD50 to ED50. Modified therapeutic drug compounds that exhibit large therapeutic indices are particularly suitable in the practice of the methods of the disclosure. The data obtained from cell culture assays and animal studies may be used in formulating a range of dosage for use in humans or other mammals. The dosage of such compounds lies preferably within a range of circulating concentrations that include the ED50 with little or no toxicity. The dosage typically varies within this range depending upon the dosage form employed, sensitivity of the patient, and the route of administration. Thus, optimal amounts will vary with the method of administration, and will generally be in accordance with the amounts of conventional medicaments administered in the same or a similar form.

The compounds of the disclosure can be administered alone, or in combination with one or more additional therapeutic agents. For example, in the treatment of cancer, the compounds can be administered in combination with compounds of the present disclosure including, but not limited to, androgen inhibitors, such as flutamide and luprolide; antiestrogens, such as tamoxifen; antimetabolites and cytotoxic agents, such as daunorubicin, fluorouracil, floxuridine, interferon alpha, methotrexate, plicamycin, mercaptopurine, thioguanine, adriamycin, carmustine, lomustine, cytarabine, cyclophosphamide, doxorubicin, estramustine, altretamine, hydroxyurea, ifosfamide, procarbazine, mutamycin, busulfan, mitoxantrone, carboplatin, cisplatin, streptozocin, bleomycin, dactinomycin, and idamycin; hormones, such as medroxyprogesterone, estramustine, ethinyl estradiol, estradiol, leuprolide, megestrol, octreotide, diethylstilbestrol, chlorotrianisene, etoposide, podophyllotoxin, and goserelin; nitrogen mustard derivatives, such as melphalan, chlorambucil, mechlorethamine, and thiotepa, steroids, such as betamethasone; and other antineoplastic agents, such as live Mycobacterium bovis, dacarbazine, asparaginase, leucovorin, mitotane, vincristine, vinblastine, and taxotere. Appropriate amounts in each case will vary with the particular agent, and will be either readily known to those skilled in the art or readily determinable by routine experimentation.

Administration of the compounds of the disclosure is accomplished by any effective route, for example, parenteral, topical, or oral routes. Methods of administration include inhalational, buccal, intramedullary, intravenous, intranasal, intrarectal, intraocular, intraabdominal, intraarterial, intraarticular, intracapsular, intracervical, intracranial, intraductal, intradural, intralesional, intramuscular, intralumbar, intramural, intraocular, intraoperative, intraparietal, intraperitoneal, intrapleural, intrapulmonary, intraspinal, intrathoracic, intratracheal, intratympanic, intrauterine, intravascular, and intraventricular administration, and other conventional means. The compounds of the disclosure having anti-tumor activity can be injected directly into a tumor, into the vicinity of a tumor, or into a blood vessel that supplies blood to the tumor.

The emulsion, microemulsion, and micelle formulations of the disclosure can be nebulized using suitable aerosol propellants that are known in the art for pulmonary delivery of the compounds. The compositions may also be modified to provide appropriate release characteristics, sustained release, or targeted release, by conventional means (e.g., coating). After compositions formulated to contain a compound and an acceptable carrier have been prepared, they can be placed in an appropriate container and labeled for use. Thus, in another aspect, the disclosure provides kits.

Vitamin E-modified nucleosides or nucleoside analogues of the disclosure are suitable for administration as oil-in-water emulsions and micelle formulations. The compounds provide for high drug loading to enable small volumes for administration.

Demonstration of Utility

The following describes a study addressing the surprising discovery that NUC013 or analogs thereof can have significantly enhanced efficacy in preventing growth and development of TP53 WT cancer cells as compared to the related decitabine analog, gemcitabine or 5-azacytidine.

As indicated herein, aberrant DNA methylation is an epigenetic mechanism that can inactivate the expression of genes that suppress tumorigenesis, such as TP53. The molecular mechanism of silencing gene expression appears to be due to the attachment of 5-methylcytosine binding proteins to the methylated promoter, which blocks the action of transcription factors. Momparler R L., “Epigenetic therapy of cancer with 5-aza-2′-deoxycytidine (decitabine).” Semin Oncol. 32:443-451 (2005), incorporated herein by reference in its entirety. Studies have shown that the pyrimidine analog decitabine induced more apoptosis in TP53 null cells than in TP53 WT cells, leading to the hypothesis that partial inhibition of DNA methylation by decitabine may cause lethality for p53 deficient cells but not WT cells. See, e.g., Nieto M, et al., “The absence of p53 is critical for the induction of apoptosis by 5-aza-2′-deoxycytidine.” Oncogene. 23(3):735-43 (2004), Leonova K I, et al., “p53 cooperates with DNA methylation and a suicidal interferon response to maintain epigenetic silencing of repeats and noncoding RNAs.” Proc Natl Acad Sci USA. 110(1):E89-98 (2013), and Yi L, et al., “Selected drugs that inhibit DNA methylation can preferentially kill p53 deficient cells. Oncotarget.” 5(19):8924-36 (2014), each of which is incorporated herein by reference in its entirety. The present studies addressed whether another pyrimidine analog, NUC013 (2′,2′-difluoro-5-aza-2′-deoxycytidine), had comparable activity to decitabine. The results surprisingly demonstrated that, compared to decitabine, NUC013 had at least as much activity in treated TP53 null cells, but had significantly more activity than decitabine in the treatment of TP53 WT cells.

The NCI-60 Cell Line Panel was assembled by the National Cancer Institute as an in vitro anticancer drug screen. The panel comprises human cancer cell lines representing nine different tissues of origin: breast, colon, central nervous system, renal, lung, melanoma, ovarian, prostate, and hematogenous. These cell lines are very well characterized, and their TP53 status (as either null/mutant or WT) has been reported. See, e.g., Leroy et al. “Analysis of TP53 Mutation Status in Human Cancer Cell Lines: a Reassesment” Hum Mutat 2014 35(6): 756-765.

As of early 2007, all compounds submitted to the NCI 60 Cell Panel have been tested initially at a single dose (10 μM). Hence, it is only possible to compare activity at 10 μM; for decitabine, whether the log GI50 is <−5.0 M or ≥−5.0 M, while for NUC013 whether at 10 μM percent growth is ≥50% or <50%. Table 1 below, provides the TP53 status of the cell lines in the NCI 60 cell line panel.

Using the cut-off of 10 μM, it is possible to summarize and compare the activity of decitabine and NUC013 in the NCI 60 Cell Line Panel and determine what influence, if any, the presence of TP53 WT has on growth inhibition by decitabine or NUC013 in 2×2 contingency tables. Only cell lines that were tested for both decitabine and NUC013 are listed in this table, i.e., 57 cell lines, as the composition of the Panel has undergone some changes with time. Fifty-five cell lines have a reported TP53 status (either wild type or null/mutant). NUC013 has been shown to inhibit the growth of hematogenous tumor cell lines by more than 50% at 10 μM and at least one cell line from all solid tumor tissues tested in the NCI 60 Cell Line Panel, including breast, colon, central nervous system, renal, lung, melanoma, ovarian and prostate.

TABLE 1
Comparison of growth inhibitory activity of decitabine and
NUC013 in the NCI 60 Cell Line Panel. Activity of NUC013
was measured at 10 μM. Growth of <50% implies that the GI₅₀
is <10⁻⁵M and, conversely, growth ≥ 50% that the GI₅₀ is ≥10⁻⁵M.
Cells in the table were shaded in dark grey for GI₅₀ ≥ 10⁻⁵M or
growth ≥ 50%, and in light gray if GI₅₀ < 10⁻⁵M or growth < 50%.

| | TP53 Status | Decitabine | NUC013 |
|---|---|---|---|
| | (0) Null/mutant | Log | Growth (%) |
| Panel/Cell line | (1) Wild type | GI₅₀ (−) M | at 10 μM |
| Leukemia | | | |
| CCRF-CEM | 0 | 4.8 | 23.67 |
| HL-60 | 0 | 4.2 | 25.23 |
| K-562 | 0 | 4.0 | 65.66 |
| MOLT-4 | 0 | 4.2 | 35.63 |
| RPMI-8226 | 0 | 3.9 | 39.24 |
| SR | 1 | 5.7 | 14.99 |
| Lung-NSCL | | | |
| A549/ATCC | 1 | 4.6 | 37.91 |
| EKVX | 0 | 3.8 | 86.06 |
| HOP-92 | 0 | 3.5 | 34.34 |
| NCI-H226 | 0 | 3.7 | 52.34 |
| NCI-H23 | 0 | 3.7 | 30.61 |
| NCI-H322M | 0 | 3.7 | 70.00 |
| NCI-H460 | 1 | 5.2 | 9.34 |
| NCI-H522 | 0 | 3.9 | 86.91 |
| Colon cancer | | | |
| HCC-2998 | 0 | 3.8 | 60.44 |
| HCT-116 | 1 | 3.7 | 29.84 |
| HCT-15 | 0 | 5.7 | 52.19 |
| HT29 | 0 | 3.8 | 70.09 |
| KM12 | 0 | 3.7 | 61.18 |
| SW-620 | 0 | 4.0 | 51.21 |
| CNS cancer | | | |
| SF-268 | 0 | 3.6 | 65.39 |
| SF-295 | 0 | 3.7 | 77.93 |
| SF-539 | 0 | 3.4 | 22.25 |
| SNB-19 | 0 | 3.4 | 58.88 |
| SNB-75 | 0 | 3.7 | 64.95 |
| Melanoma | | | |
| LOX IMVI | 1 | 3.7 | 34.53 |
| MALME-3M | 1 | 3.7 | 67.90 |
| M14 | 0 | 5.0 | 15.55 |
| MDA-MB-435 | 0 | 5.9 | 53.04 |
| SK-MEL-2 | 0 | 3.9 | 104.47 |
| SK-MEL-28 | 0 | 3.7 | 78.46 |
| SK-MEL-5 | 1 | 3.7 | 70.84 |
| UACC-257 | 1 | 4.4 | 77.54 |
| UACC-62 | 1 | 3.7 | 47.29 |
| Ovarian cancer | | | |
| IGROV-1 | 0 | 3.7 | 56.77 |
| OVCAR-3 | 0 | 3.7 | 70.56 |
| OVCAR-4 | 0 | 4.0 | 94.88 |
| OVCAR-5 | 1 | 3.6 | 86.95 |
| OVCAR-8 | 0 | 4.6 | 49.95 |
| NCI/ADR-RES | 0 | 3.5 | 42.13 |
| SK-OV-3 | 0 | 3.7 | 71.09 |
| Renal cancer | | | |
| 786-0 | 0 | 5.0 | 26.29 |
| A498 | 1 | 3.5 | 38.04 |
| ACHN | 1 | 4.2 | 7.28 |
| CAKI-1 | 1 | 3.5 | 27.86 |
| RXF 393 | 0 | 3.6 | 44.97 |
| SN12C | 0 | 3.7 | 51.65 |
| TK-10 | 0 | 4.7 | 91.13 |
| UO-31 | 1 | 3.4 | 39.00 |
| Prostate cancer | | | |
| PC-3 | 0 | 5.0 | 63.57 |
| DU-145 | 0 | 4.8 | 43.48 |
| Breast cancer | | | |
| MCF7 | 1 | 3.8 | 20.74 |
| MDA-MB-231 | 0 | 4.7 | 79.98 |
| HS 578T | 0 | 5.6 | 90.37 |
| BT-549 | 0 | 5.2 | 53.63 |

Dichotomizing drug activity into 50% growth inhibition at (≥10 μM) and (<10 μM), it is possible to summarize and compare the activity of decitabine and NUC013 in the NCI-60 Cell Line Panel and determine what influence, if any, the presence of TP53 WT has on growth inhibition by the drugs in a 2×2 contingency table.

Fisher's exact test is a statistical significance test used for the analysis of contingency tables. In the case of activity of decitabine, the p value is 0.66, confirming a very high probability of a true null hypothesis, i.e., that TP53 status has no effect on decitabine GI₅₀. This statistical analysis is compatible with the experimental data generated by Nieto and others (supra), that decitabine is not particularly effective in inducing apoptosis in TP53 WT cells.

TABLE 2
Allocation of cell lines of NCI 60 cell panel treated with
decitabine by GI50 and TP53 status.

| | TP53 | |
|---|---|---|
| NCI 60 Cell Panel | (null/mutant) | TP53 (WT) |
| >50% growth inhibition at 10 μM | 4 | 2 |
| ≤50% growth inhibition at 10 μM | 36 | 13 |

Fischer exact two-tail, p = 0.66

In contradistinction to the data presented in Table 2 for decitabine, data in Table 3 demonstrate that there is a statistically significant association between TP53 status and cell growth inhibition for NUC013 and that there is a very low probability (1.3%) that this association between TP53 WT status and NUC013 activity is due to chance.

TABLE 3
Allocation of cell lines in NCI 60 cell panel treated with
NUC013 by GI₅₀ and TP53 status.

| | TP53 | |
|---|---|---|
| NCI 60 Cell Panel | (null/mutant) | TP53 (WT) |
| >50% growth inhibition at 10 μM | 13 | 11 |
| ≤50% growth inhibition at 10 μM | 27 | 4 |

Fischer exact two-tail, p = 0.013

Another way to look at the data is that while a comparison of NUC013 efficacy versus decitabine in TP53 null/mutant cell lines is statistically significant (p=0.027, Fisher's exact test, two-tailed), the results in TP53 WT cell lines reach even more stringent levels of statistical significance (p=0.0025, Fisher's exact test, two-tailed).

Confirmation of such differences in activity between NUC013 and decitabine were further demonstrated in other TP53 WT cell lines that are not part of the NCI 60 Cell Panel indicated in Table 1. Two examples of additional comparisons are provided below for colon cancer cell lines (TP53 WT colon cancer cell line Ls174T and TP53 WT colon cancer cell line LoVo).

In these assays, two 96 well plates of each cell line were seeded with 5,000 cells per well in a total volume of 50 pL per well and left incubating overnight. The following day, the cells were exposed to different concentrations of NUC013. At the end of a 72-hour exposure period, the plates were removed for CellTiter-Glo® assay. Luminescence was recorded on a Synergy 4.0.

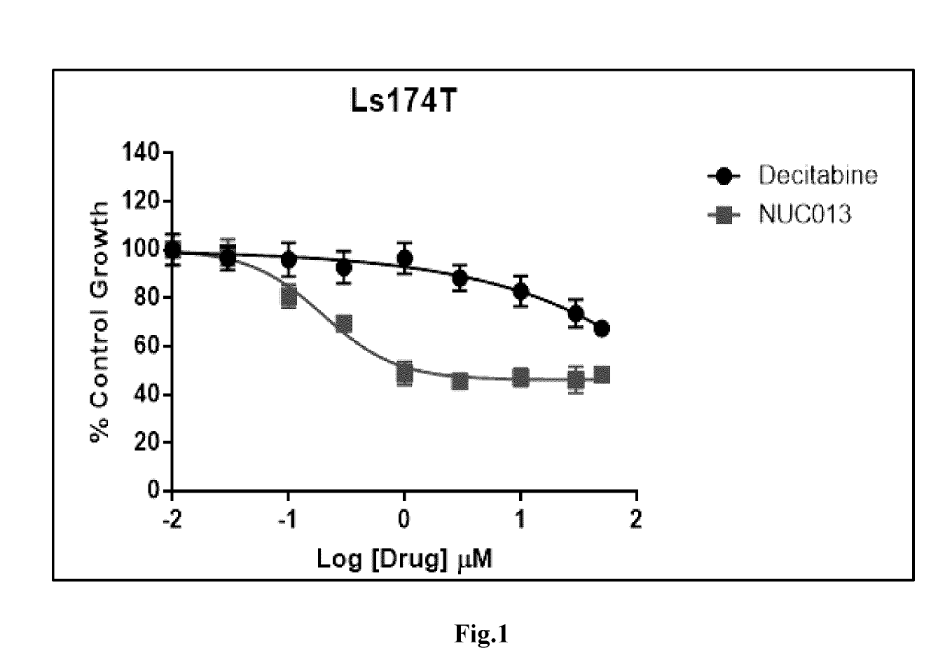

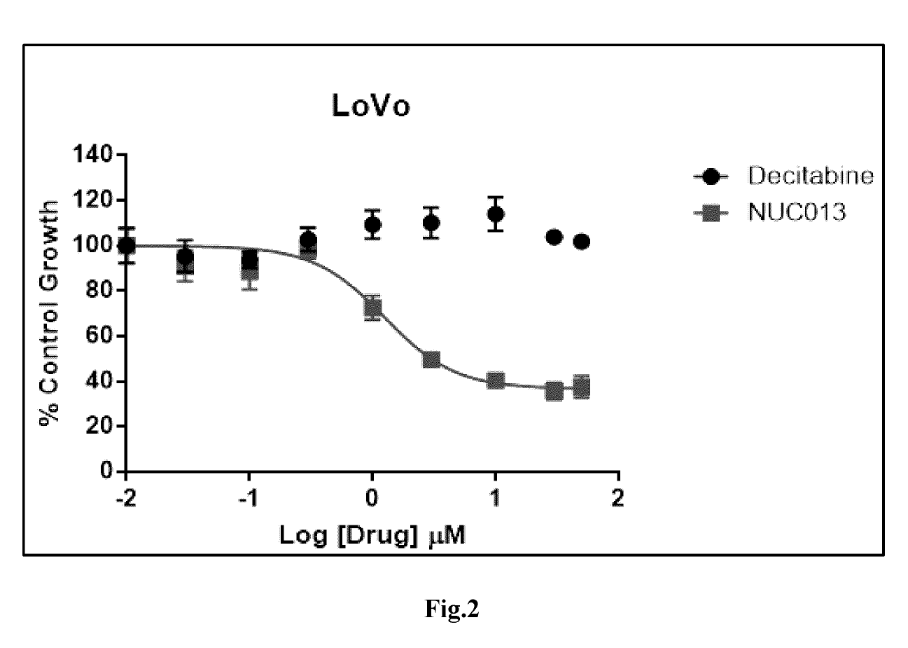

As illustrated in FIGS. 1 and 2, the GI50 is greater than 50 μM for gemcitabine in both the Ls174T and LoVo cell lines, while for NUC013 the GI50 is 1.3 μM in the Ls174T cell line and 3.0 μM for the LoVo cell line.

It is highly likely that the remarkable differences in activity illustrated in FIGS. 1 and 2 are related to the structural differences between decitabine and NUC013; i.e., the presence of the modified sugar from gemcitabine in the case of NUC013, as opposed to the natural sugar in the case of decitabine. Many 2′-substituted-2′-deoxynucleotides have been shown to be potent mechanism based RNR inhibitors. Detailed studies on 2-fluoro, 2-chloro and 2-azido derivatives have provided the basis for a general mechanism of inhibition by these substrate analogs [Wang J, Lohman G J, Stubbe J. Enhanced subunit interactions with gemcitabine-5′ diphosphate inhibit ribonucleotide reductases. Proc Natl Acad Sci USA. 2007; 104(36):14324-14329 incorporated herein by reference in its entirety].

Gemcitabine has been shown to result in the inhibition of RNR and in particular p53R2. Wang J, et al., “Mechanism of inactivation of human ribonucleotide reductase with p53R2 by gemcitabine 5′-diphosphate.” Biochemistry. 48(49):11612-21 (2009), incorporated herein by reference in its entirety. The p53R2 gene encodes a small subunit of RNR and has been identified as a p53-inducible gene. Among other activities, p53R2 provides deoxynucleotides in response to p53 activation. p53R2 has also been shown to be inducible by decitabine. Link P A, et al., “p53-inducible ribonucleotide reductase (p53R2/RRM2B) is a DNA hypomethylation-independent decitabine gene target that correlates with clinical response in myelodysplastic syndrome/acute myelogenous leukemia.” Cancer Res. 68(22):9358-66 (2008), incorporated herein by reference in its entirety. More recently, expression of p53R2 has been associated with cancer progression and resistance to therapy. See, e.g., Yousefi B. et al., “Akt and p53R2, partners that dictate the progression and invasiveness of cancer.” DNA Repair (Amst). 22:24-29 (2014), Qi J. J. et al., “E2F1 regulates p53R2 gene expression in p53-deficient cells.” Mol Cell Biochem. 399(1-2):179-88 (2015), and Matsushita S. et al., “Tp53R2 is a prognostic factor of melanoma and regulates proliferation and chemosensitivity of melanoma cells.” J Dermatol Sci. 68(1):19-24 (2012), each incorporated herein by reference in its entirety.

The inhibition of RNR by gemcitabine leads to depletion of deoxycytidine triphosphate (dCTP), a potent feedback inhibitor of deoxycytidine kinase (dCK), leading to a more efficient phosphorylation of gemcitabine. Moreover, since gemcitabine competes with dCTP, a decrease in dCTP pools increases incorporation of gemcitabine into DNA, a mechanism that has been described as self-potentiation. Mini E. et al., “Cellular pharmacology of gemcitabine.” Ann Oncol. 17 Suppl 5:v7-12 (2006) incorporated herein by reference in its entirety.

There are two approaches to determining whether a compound inhibits RNR. The more common approach is to synthesize a nucleoside diphosphate and then determine whether it inhibits RNR or p53R2. This approach would be technically very difficult with NUC013 because the diphosphate is likely to be quite unstable based on data generated with 5-azacytidine [Zielinski W S, Sprinzl M. Chemical synthesis of 5-azacytidine nucleotides and preparation of tRNAs containing 5-azacytidine in its 3′-terminus. Nucleic Acids Research. 1984; 12(12):5025-5036 incorporated herein by reference in its entirety] and experience synthesizing decitabine monophosphate. The other approach is to expose cells in tissue culture to the drug and then attempt to determine whether deoxynucleotide synthesis is inhibited when compared to control cells. Measurement of deoxynucleotides may be done by radioimmunoassay or by HPLC. The disadvantage of a radioimmunoassay is that it can only measure a single deoxynucleotide triphosphate (dNTP) at a time and typically requires a tritiated nucleotide, but it is the preferred method for measuring dCTP [Piall E M, Aherne G W, Marks V. The quantitative determination of 2′-deoxycytidine-5′ triphosphate in cell extracts by radioimmunoassay. Anal Biochem. 1986; 154(1):276-281 incorporated herein by reference in its entirety]. On the other hand, HPLC can measure multiple nucleotides without requiring radiolabeling, but has been noted to quantitate poorly dC nucleotides, possibly as an artifact of an influx of dCTP from the medium [Bianchi V, Pontis E, Reichard P. Changes of deoxyribonucleoside triphosphate pools induced by hydroxyurea and their relation to DNA synthesis. J Biol Chem. 1986; 261(34): 16037-16042 incorporated herein by reference in its entirety]. This approach was used to determine RNR inhibition by NUC013.

Briefly, HeLa cells (TP53 null cells) were grown in the presence or absence of drug, in this case gemcitabine or NUC013. At the end of a 16 h incubation, the medium was aspirated, cells monolayers washed twice with PBS, and used for nucleotide extraction. The nucleotides were extracted from cell monolayers by the addition of ice-cold 80% acetonitrile (ACN) for 1 h. The extracts were centrifuged to remove cellular debris and loaded on a SAX column (100 mg, Supelco) pre-conditioned with methanol, water and ACN. Once the sample was completely effused, the cartridge was washed with 3 ml 80% ACN and 3 ml water and eluted with 1M KCl. The eluent was filtered through a 0.45 μm filter membrane (Roth) and analyzed by HPLC. Peak identification of the different nucleoside mono-, di-, and triphosphates, was made from their characteristic UV absorption spectra and retention times compared with those of a mixture of standards run immediately before cell extracts. The area of individual peaks was measured using ChemStation software (Agilent).

Results were as follows:

TABLE 4
Concentration of nucleotides from HeLa cell extracts comparing
treatment with 50 μM gemcitabine or NUC013 vs
control replicates T1 and T2, where A: adenine,
G: guanine, T: thymidine, U: uridine, d:
deoxy, DP: diphosphate, TP: triphosphate and AU:
absorbance units. T1 and T2 are 2 replicates of drug free controls.

| | | | Gemcitabine | NUC013 |
|---|---|---|---|---|
| | Control, T1 | Control, T2 | (50 μM) | (50 μM) |
| dATP, peak area, | 15.8 | 15.3 | 0 | 10.2 |
| AU | | | | |
| dGDP, peak area, | 11.3 | 10.3 | 7.95 | 5.6 |
| AU | | | | |
| dTDP, peak area, | 20.6 | 18.2 | 43.4 | 18.65 |
| AU | | | | |
| dUTP, peak area, | 16.8 | 35.2 | 5.3 | 8.8 |
| AU | | | | |
| ADP | 14,052 | 14,188 | 15,639 | 13,845 |
| ATP | 22,163 | 17,489 | 26,296 | 21,335 |
| GTP | 2,003 | 1,151 | 2,036 | 2,006 |
| CTP | 1,209 | 848 | 1,297 | 1,279 |
| UTP | 4,011 | 4,146 | 4,801 | 4,070 |

Table 4 shows that neither gemcitabine nor NUC013 affected concentrations of ribonucleotides. However, lower concentration of deoxynucleotides are noted from lysates from cells treated with gemcitabine or NUC013, in particular of dATP, dGDP and dUTP. dTDP appears to be unaffected by presence of NUC013, perhaps as a result of the presence of a salvage pathway, while the increase in levels of dTDP with gemcitabine may be due to co-elution with gemcitabine-DP. These results are compatible with RNR inhibition by both gemcitabine and NUC013. It should be noted that the relative effectiveness of the compounds as RNR inhibitors may vary depending on the cell line used for the assay and that HeLa cells are TP53 null.

The p53R2 gene encodes a small subunit of RNR and has been identified as a p53-inducible gene. While gemcitabine does not induce p53R2, it has been shown to result in its inhibition [Wang J, Lohman G J, Stubbe J. Mechanism of inactivation of human ribonucleotide reductase with p53R2 by gemcitabine 5′-diphosphate. Biochemistry. 2009; 48(49):11612-11621]. Conversely, p53R2 has been shown to be inducible by decitabine and among other activities, p53R2 provides deoxynucleotides in response to p53 activation [Link P A, Baer M R, James S R, Jones D A, Karpf A R. p53-inducible ribonucleotide reductase (p53R2/RRM2B) is a DNA hypomethylation-independent decitabine gene target that correlates with clinical response in myelodysplastic syndrome/acute myelogenous leukemia. Cancer Res. 2008; 68(22):9358-9366]. More recently, expression of p53R2 has been associated with cancer progression and resistance to therapy [Yousefi B, Samadi N, Ahmadi Y. Akt and p53R2, partners that dictate the progression and invasiveness of cancer. DNA Repair (Amst). 2014; 22:24-29. Qi J J, Liu L, Cao J X, An G S, Li S Y, Li G et al. E2F1 regulates p53R2 gene expression in p53-deficient cells. Mol Cell Biochem. 2015; 399(1-2): 179-188. Matsushita S, Ikeda R, Fukushige T, Tajitsu Y, Gunshin K, Okumura H et al. Tp53R2 is a prognostic factor of melanoma and regulates proliferation and chemosensitivity of melanoma cells. J Dermatol Sci. 2012; 68(1):19-24]. RNR is a complex between two proteins: the large catalytic protein R1 that contains the allosteric sites and the smaller protein R2. Both proteins are transcriptionally activated during early S-phase and are present in roughly equal amounts to deliver the dNTP required for DNA replication. R2 is degraded during late mitosis and thus postmitotic quiescent cells are essentially devoid of R2 but retain some R1. In postmitotic resting cells, only p53R2 can act as the functioning small subunit of RNR because it is not degraded in mitosis. After DNA damage, p53R2 is transcriptionally activated by p53 and is translocated into the nucleus but has also been shown to be present in the cytosol [Pontarin G, Ferraro P, Bee L, Reichard P, Bianchi V. Mammalian ribonucleotide reductase subunit p53R2 is required for mitochondrial DNA replication and DNA repair in quiescent cells. Proc Natl Acad Sci USA. 2012; 109(33):13302-13307].

Decitabine has been thought of as an S-phase specific agent. However, it has recently been shown that the cell cycle dependence of decitabine is not absolute. Its secondary, epigenetic effects are replication dependent, but its primary effect, DNMT1 enzyme depletion, occurs immediately with elimination half-lives that are unrelated to the fraction of cells in S-phase. Thus, DNA repair and remodeling activity in arrested, confluent cells may be sufficient to support the primary molecular action of decitabine, while its secondary, epigenetic effects require cell cycle progression through S-phase [Al-Salihi M, Yu M, Burnett D M, Alexander A, Samlowski W E, Fitzpatrick F A. The depletion of DNA methyltransferase-1 and the epigenetic effects of 5-aza-2′deoxycytidine (decitabine) are differentially regulated by cell cycle progression. Epigenetics. 2011; 6(8): 1021-1028]. In one embodiment, the ability of NUC013 to inhibit DNMT at a concentration below that causing substantial cell growth arrest favors the expression of its epigenetic effects.

The inhibition of RNR by gemcitabine has been reported to lead to depletion of dCTP, a potent feedback inhibitor of deoxycytidine kinase, leading to a more efficient phosphorylation of gemcitabine. Moreover, since gemcitabine competes with dCTP, a decrease in dCTP pools increases incorporation of gemcitabine into DNA, a mechanism that has been described as self-potentiation [Mini E, Nobili S, Caciagli B, Landini I, Mazzei T. Cellular pharmacology of gemcitabine. Ann Oncol. 2006; 17 Suppl 5:v7-12 incorporated herein by reference in its entirety]. This same mechanism of action is hypothesized to apply to NUC013 and inhibition of RNR and p53R2. Thus, NUC013 can inhibit dNTP synthesis not only during S phase but throughout the cell cycle. In contradistinction, restoration of p53 WT function by decitabine [Zhu W G, Hileman T, Ke Y, Wang P, Lu S, Duan W et al. 5-aza-2′-deoxycytidine activates the p53/p21Waf1/Cip1 pathway to inhibit cell proliferation. J Biol Chem. 2004; 279(15): 15161-15166 incorporated herein by reference in its entirety] and induction of p53R2 should increase the pool of dCTP, thereby decreasing the incorporation of decitabine during the resting phase, a mechanism that might be described as “self-antagonism.” This self-antagonism may be one reason for the lesser activity of decitabine when compared to NUC013.

The remarkable activity of NUC013 in treating TP53 WT tumors is unique to NUC013, and is unlikely to be replicable by combination treatment with gemcitabine and decitabine, and even less so in a combination of gemcitabine with 5-azacytidine. In the case of 5-azacytidine, gemcitabine inhibits RNR and RNR is essential for the reduction of 5-azacytidine to 5-aza-2′-deoxycytidine. Because gemcitabine and decitabine are both cytidine analogs, they would be competing with each other for the many of the same enzymatic systems necessary for their incorporation as deoxycytidine triphosphate analogs in DNA, including polymerase a. Furthermore, administration of both gemcitabine and decitabine would put a patient at risk from side effects from two chemotherapeutic agents, thus rendering such a therapeutic regime unlikely.

In summary, while active against TP53 null/mutant cell lines, the DNMTI NUC013 has been shown to have a unique ability to treat TP53 WT tumor cell lines.

Demonstration of Safety Profile

The following describes a study comparing the efficacy and toxicity of decitabine and NUC013 in mice xenograft models, and demonstrates the surprising discovery that NUC013 is significantly less toxic than decitabine in the treatment of animals implanted with a TP53 WT cell line, while simultaneously exhibiting greater efficacy for preventing tumor growth.

Minor structural changes to nucleosides make assumptions about toxicity difficult because of the central role of nucleosides in cellular function. An example is fialuridine (FIAU; 1-(2-deoxy-2-fluoro-1-D-arabinofuranosyl)-5′-iodouracil, which is a nucleoside analogue that differs from uridine by the substitution of a fluorine for a hydrogen at the 2′ position of the sugar and an iodine for a hydrogen at the 5′ position of the base. These structural changes relating to two substitutions (which is similar to the extent of differences between the structure of NUC013 and decitabine), resulted in unexpected toxicity. In June 1993, a clinical trial of FIAU for the treatment of hepatitis B was abruptly terminated when one of the 15 out-patients participating in the National Institutes of Health (NIH) study was suddenly hospitalized with liver failure. Although all of the remaining patients were contacted and told to stop taking their medication, six more developed severe toxicity in the next few weeks. Five patients died, and two others were probably saved from death only by liver transplantation. This illustrates the potential for toxicity risks in nucleoside analogs as disease therapeutics, where minor variations can result in large increases in toxicity.

In animal models, NUC013 has demonstrated greater safety than the approved drug decitabine. This improved safety profile was unexpected because, in addition to targeting DNA methyl transferase, as does decitabine, NUC013 also targets ribonucleotide reductase (RNR). Such an enhanced spectrum of activity would have been expected to also lead to a greater risk of toxicity. However, this is not the observed result in animal tumor models. For example, in xenograft models in nude mice implanted with HL-60 (leukemia, TP null) or LoVo (colon cancer, TP WT), NUC013 has demonstrated greater efficacy and less toxicity than decitabine.

Specifically, at a dose of 5 mg/kg per day for three consecutive days a week for three weeks, decitabine was not more effective than saline and was not tolerated by nude mice implanted with HL-60 (hazard ratio (HR)=0.77, p=0.62) (the hazard ratio can be interpreted as the chance of an event occurring in the treatment arm (defined as death, ulcerated tumor or tumor >4 g) divided by the chance of the event occurring in the saline control arm). Four of ten mice treated with decitabine were dead or moribund prior to the conclusion of the study. These results can be contrasted with treatment with NUC013 at an equimolar dose (5.8 mg/kg), which demonstrated trends towards improved survival (HR=0.46, p=0.019) when compared to the saline control with no animals removed from the study for death or moribund. At a dose of 20 mg/kg of NUC013, mice treated with NUC013 had improved survival versus saline control mice (HR=0.26, p=0.032) at the conclusion of the study.

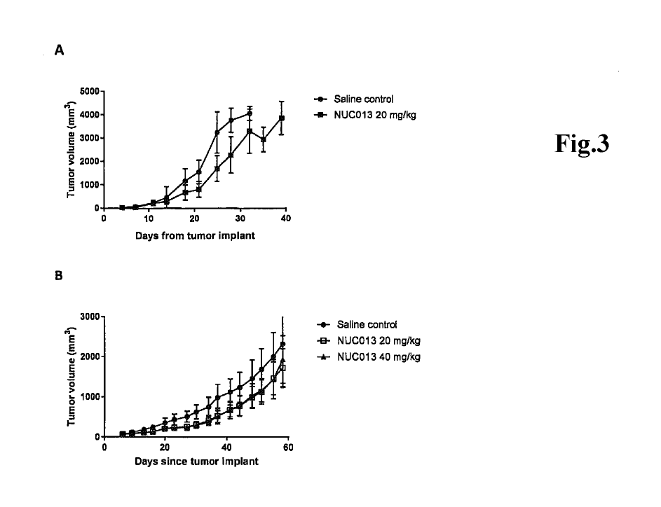

While tumor weights were not significantly different between animals treated at 5.8 mg/kg and saline control, at 20 mg/kg significant decreases in tumor volume (p<0.05, Mann-Whitney U test, two-tailed) were noted on study days 18 through 28, illustrated in FIG. 3A.

In the study with mice implanted with LoVo treated with 5 mg/kg of decitabine eight of ten died and the remaining two (of ten) had to be euthanized because of tumor ulceration within 35 days of tumor implantation. These results were significantly worse than the survival of mice in the saline control group (median survival decitabine 31 days, median survival saline over 60 days, HR=26.89, p<0.0001) where 4/10 mice were euthanized for tumors greater than 4 g but the remaining 6/10 survived until the end of the experiment. At 5.8 mg/kg, NUC013 showed no difference in survival compared to saline controls, however, eight days following the end of treatment, mean tumor weight was lower by 24.3% compared to controls (p=0.048) 8 days following the end of treatment. At doses of 20 mg/kg and 40 mg/kg, mean tumor volumes were significantly lower in both treated groups (p<0.05, Mann-Whitney U test, two-tailed) on study days 9 through 51 compared to saline control, illustrated in FIG. 3B.

Furthermore, the maximum tolerated dose (MTD) of NUC013 has not yet been established in nude mice, but it has been demonstrated to be greater than 120 mg/kg when NUC013 is administered intravenously for three consecutive days a week for three weeks, suggesting a significantly better therapeutic index than decitabine. Such relative lack of toxicity may allow treatment of tumors relatively resistant to NUC013, such as pancreatic cancer (e.g., the GI50 of cell line CFPAC-1 is 53.8 μM).

Treatment with a prodrug of NUC013 (NUC041, 3′,5′-di(trimethylsilyl)-2′,2′-difluoro-5-aza-2′-deoxycytidine) has also shown activity against nude mice implanted with HL-60 or LoVo. As NUC013 is subject to hydrolysis, NUC041 is designed to be formulated in a hydrophobic matrix. Original experiments with NUC041 in an oil in water emulsion with a droplet size of less than 100 nm did not demonstrate a pharmacokinetic advantage over NUC013. Nonetheless, at a dose of 31 mg/kg (equimolar to a dose of 20 mg/kg of NUC013) administered intravenously, NUC041 treated HL-60 mice had a trend toward improved survival (HR=0.51, p=0.26) compared to saline controls. In mice implanted with LoVo, tumor weights in mice treated with NUC041 were 26.6% less than saline controls (p=0.020) 8 days following the end of treatment. More recently, formulation of NUC041 in a phospholipid gel depot administered subcutaneously to mice demonstrated dramatic improvements in pharmacokinetic parameters. While the half-life of NUC013 administered IV to mice is 20 minutes, the time of maximum mean concentration (Tmax) of NUC013 released from NUC041 in gel is at 4 hours and the drug is still present in circulation 24 hours following subcutaneous injection.

TABLE 5
Pharmacokinetic Parameters Derived from Mean
Concentrations of NUC041 and NUC013 in Plasma
Following SC Administration of NUC041 (100 mg/kg)
to Mice.

| | Cₘₐₓ^{a} | Tₘₐₓ^{b} | AUCₗₐₛₜ^{c} |
|---|---|---|---|
| Analyte | (ng/mL) | (hr) | (hr · ng/mL) |
| NUC041 | 21.7 | 0.5 | 20.0 |
| NUC013 | 436 | 4 | 1939 |

^{a}Maximum observed mean concentration in plasma
^{b}Time of maximum mean concentration was observed in plasma
^{c}Area under the plasma concentration versus time curve from 0 to the last sampling time the analyte was quantifiable in plasma

Optimization of the formulation for NUC041 has yet to be completed and this is expected to further enhance the observed pharmacology and therapeutic efficacy of the prodrug.

Thus, it is demonstrated that NUC013 and the prodrug of NUC013, NUC041, are more effective than decitabine in xenograft tumor models, of both TP53 WT and TP53 null cell lines. Furthermore, its relative lack of toxicity allows higher doses to be employed to enhance efficacy in vivo.

The following example is provided for the purpose of illustrating aspects of the disclosure and is not to be construed as limiting to the disclosed invention.

### Example 1

### Preparation of 2′,2′-Difluoro-5-azadeoxycytidine (NUC013)

Two alternative pathways, shown in Scheme I, were used to prepare 2′,2′-difluoro-5-aza-deoxycytidine (Compound IV) (NUC013).

### Pathway 1—Step A—Preparation of 3′,5′-dibenzoyl-2′,2′-difluoro-5-azadeoxycytidine (Compound III) Via 1-bromo-2-deoxy-2,2-difluoro-D-ribofuranosyl-3,5-dibenzoate (Compound I)

(1) The starting material 2-deoxy-2,2-difluoro-D-ribofuranosyl-3,5-dibenzoate known in the art (Chou, et. al., Synthesis 1992, v.p. 565-570) was converted to 1-bromo analog by a method similar to the one described in International Patent Application WO 2006/070985. The starting ribose (3.9 g, 10.3 mmol) was dissolved in 31 ml of toluene and dry triethylamine (1.43 ml, 10.3 mmol) was added. The solution was cooled to 0° C. and diphenyl phosphoryl chloride (2.64 ml, 12.3 mmol) in 8 ml of toluene was added during 15 minutes. The reaction mixture was warmed up to room temperature and incubated for 3.5 hours. The reaction was quenched by addition of 1 M HCl (10.2 ml), the toluene layer was separated and aqueous layer was extracted back with 10 ml ether. The organic layers were combined, extracted consequently with water, saturated NaHCO₃, and saturated NaCl (each 20 ml) and dried over MgSO₄. The solvents were removed and the product was isolated by flash chromatography on silica gel in hexane-ethyl acetate gradient. Yield 4.6 g, 73%.

(2) To the intermediate diphenylphosphate analog (4.6 g, 7.5 mmol) prepared as described in Step (1), was added HBr in acetic acid (30%, 16.2 ml, 81.3 mmol) and the reaction mixture was incubated for 6.5 hours at room temperature. The reaction mixture was diluted with 80 ml of methylene chloride and extracted twice with ice water, saturated NaHCO₃, and saturated NaCl (each 100 ml). The organic layer was dried with MgSO₄, filtered and evaporated to yield 1-bromo-2-deoxy-2,2-difluoro-D-ribofuranosyl-3,5-dibenzoate (Compound I, 3.1 g, 93%), which is a 9:1 mixture of a and P isomers.

(3) 5-Azacytosine (98%, Aldrich, 5.0 g, 44.8 mmol) and ammonium sulfate (25 mg) were suspended in hexamethyldisilazane (25 ml) and chlorotrimethylsilane (0.2 ml) was added. The reaction mixture was heated at the reflux for 17 hours. The clear solution was cooled, evaporated, coevaporated twice with dry xylene and vacuum dried to yield whitish solids of the silylated 5-azacytosine (˜7 g) which is used in whole for the next glycosylation step.

(4) For glycosylation step 1-bromo-2-deoxy-2,2-difluoro-D-ribofuranosyl-3,5-dibenzoate (Compound I) (0.78 g, 1.77 mmol), prepared as described in Step (2), was dissolved in 4 ml anisole and transferred to the silylated 5-azacytosine solids prepared as in Step (3). The suspension was heated to 150° C. and 2 ml of anisole was added to complete dissolution of all solids. After four hours, the reaction mixture was cooled and SnCl₄ (0.2 ml, 2 mmol) was added. The reaction mixture was reheated to 150° C. for 6 hours, cooled to room temperature, quenched by addition of methylene chloride (30 ml), methanol (10 ml) and silica gel (20 g) and dried to yield 3′,5′-dibenzoyl-2′,2′-difluoro-5-azadeoxycytidine (Compound III). The resulting powder was applied on the top of the packed silica gel column and products were isolated by flash chromatography in chloroform-methanol gradient. P-Isomer was eluted first followed by a-isomer (only partial separation was achieved, yield for P-isomer 12%, a-isomer 5%). Complete isomer separation was achieved by RP HPLC on Gemini C18 5u (21.2×250 mm column) in 50 mM triethylammonium acetate (pH 7.5)-acetonitrile gradient.

NMR in CDCl₃ for compound III P-isomer: 8.12 ppm (s, 1H, H6), 7.8-8.1 (m, 4H, benzoyl), 7.4-7.6 (m, 2H, benzoyl), 7.2-7.4 (m, 4H, benzoyl), 6.38 (br.t, J=8.0 Hz, 1H, H1′), 5.65 (m, 1H, H3′), 4.70 (m, 2H, H5′), 4.58 (m, 1H, H4′). ESI MS: 473.3 [M+H]+, 471.1 [M−H]−.

### Pathway 1—Step B—Preparation of 2′,2′-difluoro-5-azadeoxycytidine (Compound IV)

3′,5′-dibenzoyl-2′,2′-difluoro-5-azadeoxycytidine (Compound III) (15 mg, 3:1 mixture of a and P isomers), prepared as in Step A, was dissolved in 2 ml of anhydrous MeOH and 1 M NaOMe in MeOH (0.1 ml) was added. After one hour at room temperature, the reaction mixture was evaporated and the deprotected isomers were isolated by RP HPLC on Gemini C18 5u (21.2×250 mm column) in 50 mM triethylammonium acetate (pH 7.5)-acetonitrile gradient. Immediately after separation fractions corresponding to P isomer were pooled and evaporated at below 10° C. to yield 0.4 mg (19%) of 2′,2′-difluoro-5-azadeoxycytidine (Compound IV).

¹H NMR in DMSO-d₆ for compound IV P-isomer: 8.48 ppm (s, 1H, H6), 7.79 (br, 2H, NH₂), 6.35 (br, 1H, 3′-OH), 6.07 t, J=8.0 Hz, 1H, H1′), 5.30 (br, 1H, 5′-OH), 4.90 (br, 1H, 5′-OH), 4.23 (br. m, 1H, H3′), 3.85 (m, 1H, H4′), 3.78 (m, 1H, H5′), 3.63 (m, 1H, H5′), UV 240 nm (sh) in 50 mM triethylammonium acetate (pH 7.5).

### Pathway 2—Step C—Preparation of 3′,5′-dibenzoyl-2′,2′-difluoro-5-azadeoxycytidine (Compound III) Via 1-methylsulfonyl-2-deoxy-2,2-difluoro-D-ribofuranosyl-3,5-dibenzoate (Compound II)

Silylated 5-azacytosine (2.3 g, 9 mmol) prepared as described in Step A(3), was dissolved in 2 ml of anisole at 130° C. 1-Methylsulfonyl-2-deoxy-2,2-difluoro-D-ribofuranosyl-3,5-dibenzoate (Compound II) (0.4 g, 0.87 mmol), prepared as described in the art (Chou, et. al., Synthesis v.p. 565-570, 1992), was dissolved in 1 ml of anisole and was added to the hot solution of the silylated 5-azacytosine. The reaction mixture was incubated at 150° C. for seven hours, cooled to room temperature, quenched by addition of 15 ml methylene chloride, 15 g silica gel and 5 ml methanol and the suspension was dried on vacuum. The resulting powder was applied on the top of the packed silica gel column and products were isolated by flash chromatography in chloroform-methanol gradient. Appropriate fractions were pooled and evaporated to yield 3′,5′-dibenzoyl-2′,2′-difluoro-5-azadeoxycytidine (Compound III) as 2:1 mixture of a- and P-isomers (100 mg, 25% yield).

### Pathway 2—Step D. Preparation of 2′,2′-difluoro-5-azadeoxycytidine (Compound IV)

3′,5′-dibenzoyl-2′,2′-difluoro-5-azadeoxycytidine (Compound III) (80 mg, 2:1 mixture of a and P isomers), prepared as in Step C, was dissolved in 2 ml of anhydrous MeOH and 1 M NaOMe in MeOH (0.1 ml) was added. After one hour at room temperature, the reaction mixture was evaporated and the deprotected isomers were isolated by RP HPLC on Gemini C18 5u (21.2×250 mm column) in 50 mM triethylammonium acetate (pH 7.5)-acetonitrile gradient. Immediately after separation fractions corresponding to P isomer were pooled, evaporated at below 10° C. to yield 2.1 mg (13%) of 2′,2′-difluoro-5-azadeoxycytidine (Compound IV).

RP HPLC retention time and spectral characteristics (i.e. 1H NMR and UV) for Compound IV were the same as in Step B. ESI MS: 265.2 [M+H]+.

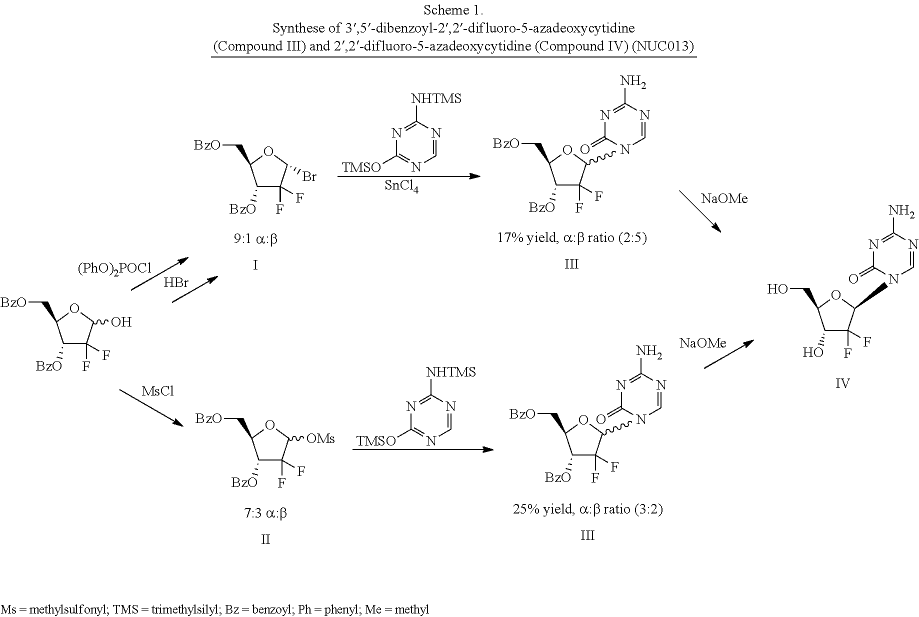

The following table 6 summarizes the structures mentioned in this patent application.

| Compound | Structure | IUPAC name |
|---|---|---|
| Cytidine | 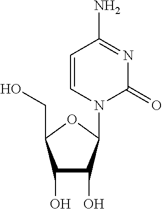 | 4-amino-1-(2R,3R,4S,5R)-3,4- dihydroxy-5- (hydroxymethyl)tetrahydrofuran-2 yl)pyrimidin-2(1H)-one |
| 5-Azacytidine | 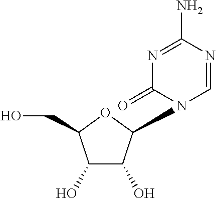 [SMILES: Nc1ncn([C@@H]2O[C@H](CO)[C@@H](O)[C@H]2O)c(=O)n1] | 4-amino-1-((2R,3R,4S,5R)-3,4- dihydroxy-5- (hydroxymethyl)tetrahydrofuran-2-yl)- 1,3,5-triazin-2(1H)-one |
| 5-azadeoxycytidine or decitabine | 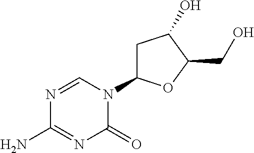 [SMILES: Nc1ncn([C@H]2C[C@H](O)[C@@H](CO)O2)c(=O)n1] | 4-amino-1-((2R,4S,5R)-4-hydroxy-5- (hydroxymethyl)tetrahydrofuran-2-yl)- 1,3,5-triazin-2(1I)-one |
| | [symbol] | |
| Gemcitabine | 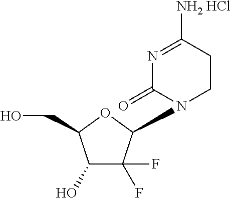 | 4-amino-1-((2R,4R,5R)-3,3-difluoro-4- hydroxy-5- (hydroxymethyl)tetrahydrofuran-2- yl)pyrimidin-2(1I)-one hydrochloride |
| 2′,2′-difluoro-5- azadeoxycytidine (NUC013) | 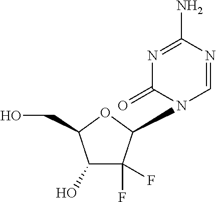 [SMILES: Nc1ncn([C@@H]2O[C@H](CO)[C@@H](O)C2(F)F)c(=O)n1] | 4-amino-1-((2R,4R,5R)-3,3-difluoro-4- hydroxy-5- (hydroxymethyl)tetrahydrofuran-2-yl)- 1,3,5-triazin-2(1H)-one |
| 2′,2′-difluoro-5,6- dihydro-5- azadeoxycytidine (NUC014) | 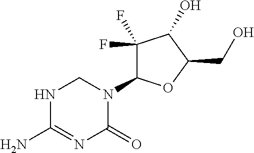 | 4-amino-1-((2R,4R,5R)-3,3-difluoro-4- hydroxy-5- (hydroxymethyl)tetrahydrofuran-2-yl)- 5,6-dihydro-1,3,5-triazin-2(1H)-one |
| | [symbol] | |
| 2′-deoxy-2′,2′- difluorozebularine (NUC019) | 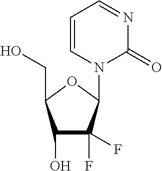 [SMILES: O=c1ncccn1[C@@H]1O[C@H](CO)[C@H](O)C1(F)F] | 1-((2R,4R,5R)-3,3-difluoro-4-hydroxy- 5-(hydroxymethyl)tetrahydrofuran-2- yl)pyrimidin-2(1H)-one |
| Zebularine | 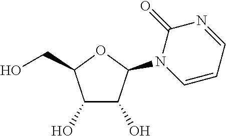 [SMILES: O=c1ncccn1[C@@H]1O[C@H](CO)[C@@H](O)[C@H]1O] | 1-((2R,3R,4S,5R)-3,4-dihydroxy-5- (hydroxymethyl)tetrahydrofuran-2- yl)pyrimidin-2(1H)-one |
| 2′,2′- difluorozebularine | 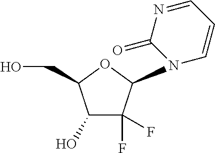 [SMILES: O=c1ncccn1[C@@H]1O[C@H](CO)[C@@H](O)C1(F)F] | 1-((2R,4R,5R)-3,3-difluoro-4-hydroxy- 5-(hydroxymethyl)tetrahydrofuran-2- yl)pyrimidin-2(1H)-one |
| 2′,3′,5′- tri(trimethylsilyl)- 5-azacytidine (NUC025) | 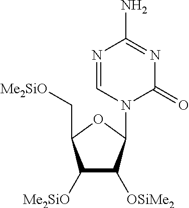 | 4-amino-1-((2R,3R,4S,5R)-3,4- dihydroxy-5- (hydroxy(trimethylsilyl)methyl)-3,4- bis(trimethylsilyl)tetrahydrofuran-2- yl)-1,3,5-triazin-2(1H)-one |
| 2′,2′-difluoro-5- azadeoxycytidine- trimethylsilyl (NUC041) 3′,5′- di(trimethylsilyl)- 2′,2′-difluoro-5-aza- 2′-deoxycytidine | 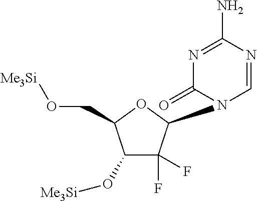 [SMILES: C[Si](C)(C)OC[C@H]1O[C@@H](n2cnc(N)nc2=O)C(F)(F)[C@@H]1O[Si](C)(C)C] | 4-amino-1-((2R,4R,5R)-3,3-difluoro-4- (trimethylsilyloxy)-5- ((trimethylsilyloxy)methyl) tetrahydrofuran-2-yl)- 1,3,5-triazin-2(1H)-one |

[symbol]

indicates data missing or illegible when filed

All cited references are incorporated herein by reference in their entirety.

While the preferred embodiment of the disclosure has been illustrated and described, it will be appreciated that various changes can be made therein without departing from the spirit and scope of the disclosure.

1. A method for inhibiting the growth of a TP53 wild-type cancer cell, comprising contacting the TP53 wild-type cancer cell with 2′,2′-difluoro-5-aza-2′-deoxycytidine or a 2′,2′-difluoro-5-aza-2′-deoxycytidine prodrug or a composition comprising 2′,2′-difluoro-5-aza-2′-deoxycytidine or a 2′,2′-difluoro-5-aza-2′-deoxycytidine prodrug and a pharmaceutically acceptable carrier, diluent or excipient.

2. The method of claim 1, wherein the 2′,2′-difluoro-5-aza-2′-deoxycytidine prodrug is a silylated compound of 2′,2′-difluoro-5-aza-2′-deoxycytidine or the 2′,2′-difluoro-5-aza-2′-deoxycytidine prodrug.

3. The method of claim 1, wherein the 2′,2′-difluoro-5-aza-2′-deoxycytidine prodrug is 3′,5′-di(trimethylsilyl)-2′,2′-difluoro-5-aza-2′-deoxycytidine.

4. The method of claim 1, wherein the 2′,2′-difluoro-5-aza-2′-deoxycytidine prodrug is a tocopherol phosphate prodrug of 2′,2′-difluoro-5-aza-2′-deoxycytidine.

5. The method of claim 1, wherein the prodrug is a tocotrienol phosphate prodrug of 2′,2′-difluoro-5-aza-2′-deoxycytidine.

6. The method of claim 1, wherein the cancer cell is from a solid tumor, a solid tumor carcinoma, non-small cell lung NSCL, colon, renal, central nervous system CNS, melanoma, ovarian, prostate, pancreatic, or breast carcinomas.

7. (canceled)

8. The method of claim 1, wherein the cancer cell is from a hematologic malignancy, leukemia, lymphoma, or multiple myeloma.

9. The method of claim 1, wherein the cell is in vitro.

10. The method of claim 1, wherein the cell is in vivo in a subject and an effective amount of the 2′,2′-difluoro-5-aza-2′-deoxycytidine or a 2′,2′-difluoro-5-aza-2′-deoxycytidine prodrug is administered to the subject, and the subject is human.

11. A method of treating a cancer characterized by having wild-type TP53, comprising administering to a subject in need thereof a therapeutically effective amount of 2′,2′-difluoro-5-aza-2′-deoxycytidine or a 2′,2′-difluoro-5-aza-2′-deoxycytidine prodrug or a composition comprising 2′,2′-difluoro-5-aza-2′-deoxycytidine or a 2′,2′-difluoro-5-aza-2′-deoxycytidine prodrug and a pharmaceutically acceptable carrier, diluent or excipient.

12. The method of claim 11, wherein the 2′,2′-difluoro-5-aza-2′-deoxycytidine prodrug is a silylated compound of 2′,2′-difluoro-5-aza-2′-deoxycytidine or the 2′,2′-difluoro-5-aza-2′-deoxycytidine prodrug.

13. The method of claim 11, wherein the 2′,2′-difluoro-5-aza-2′-deoxycytidine prodrug is 3′,5′-di(trimethylsilyl)-2′,2′-difluoro-5-aza-2′-deoxycytidine.

14. The method of claim 11, wherein the 2′,2′-difluoro-5-aza-2′-deoxycytidine prodrug is a tocopherol phosphate prodrug of 2′,2′-difluoro-5-aza-2′-deoxycytidine.

15. The method of claim 11, wherein the prodrug is a tocotrienol phosphate prodrug of 2′,2′-difluoro-5-aza-2′-deoxycytidine.

16. The method of claim 11, wherein the cancer is a solid tumor, a solid tumor carcinoma, non-small cell lung NSCLC, colon, renal, central nervous system CNS, melanoma, ovarian, renal, prostate, pancreatic, or breast carcinomas.

17. (canceled)

18. The method of claim 11, wherein the cancer is a hematologic malignancy, leukemia, lymphoma, or multiple myeloma.

19. The method of claim 11, wherein the subject is a human.

20.-38. (canceled)
